(19) United States
(12) Patent Application Publication
(10) Pub. No.: US 2019/0008396 A1
(43) Pub. Date: Jan. 10, 2019

(54) CONCAVE OPTICAL SENSORS

(71) Applicant: X-CARDIO CORP. KK, Tokyo (JP)

(72) Inventor: Ehud BARON, OKAYAMA (JP)

(21) Appl. No.: 15/750,399

(22) PCT Filed: Aug. 4, 2016
(86) PCT No.: PCT/IB16/54712

§ 371 Date: Feb. 5, 2018

Related U.S. Application Data
(60) Provisional application No. 62/201,137, filed on Aug. 5, 2015.
(60) Provisional application No. 62/276,248, filed on Jan. 8, 2016.

(51) Int. Cl. A61B 5/0205; A61B 5/024; A61B 5/1455; A61B 5/0245

(52) U.S. Cl. CPC A61B 5/0205; A61B 5/02427; A61B 5/6826; A61B 5/02438; A61B 5/0245; A61B 5/14552

(57) ABSTRACT

A concave optical sensor comprising at least one light source having at least one wavelength, wherein the light source is configured to emit light beams, and at least one light receiver responsive to the light beams emitted from the light source. Both the at least one light source and the at least one light receiver are positioned on a concave segment. The concave segment cane be an elastic segment or can further be provided with a resilient element configured to press the sensor against a body part to obtain optimal coupling and to prevent motion artifacts.

### FIELD

The present subject matter relates to physiological spectrographic and cardiovascular monitoring. More particularly, the present subject matter relates to systems and methods for obtaining improved PhotoPlethysmoGraphy (PPG) and pulse oximetry signals using concave optical sensors in various body parts.

### BACKGROUND

A high quality, signal is required for monitoring of the spectrographic and hemodynamics of the cardiovascular (or circulatory) system. Using an optical sensor consisting of light sources (such as LEDs or LASERS) and a light sensing element (e.g. photodiodes or Light-to-Frequency transducers), and then illuminating through body tissue with capillary bed, one can get a signal that changes in time in accordance with the heart-beat. Such a signal of the blood volume changes with heart beating can be translated into electrical signal of changes in light intensity. This pulsating wave generated by changes in light absorption proportional to blood volume changes is known as PhotoPlethysmoGraphy, or in short PPG. By adding additional wavelengths it is possible to measure various blood characteristics, such as the arterial blood oxygen saturation of hemoglobin. Additional wavelengths can also be used for measuring other constituents of the blood. In pulse oximetry, the light passing through the tissue is typically selected from several wavelengths that can be absorbed by the blood in an amount corresponding to the amount of the hemoglobin constituent present in the blood. The amount of light absorbed at different light wavelengths can then be used to estimate the arterial blood hemoglobin related parameters using various known algorithms.

The intensity of the light (detected by the sensor's photodetector) is altered by pulsatile changes in the volume of the arterial blood at the illuminated site, during blood pressure wave propagation. The quality of the pulse oximetry measurement depends in part on the blood perfusion characteristics of the tissue, illuminated by the light and in part on the magnitude of the pulsatile changes in the blood volume within the illuminated tissue. Pulse oximetry techniques typically utilize a tissue site that is well perfused with blood but also thin enough to shine light through it. Such sites are the fingertip, toe tip, or earlobe, through which the light can pass.

Most commercially available pulse oximeters are configured for short measurements, and use a clip type (or “crocodile”) sensor that illuminates light from one side of a boneless tissue such as a fingertip, toe tip or earlobe, and then collects it from the other side. This method is called “Transmissive” as light goes through the illuminated tissue.

Oximetry sensors are typically coupled to monitoring systems, for instance via suitable cables. For example, if such sensors are used for vital sign monitoring of patients in the hospital. Accordingly, such continuous monitoring typically requires the patient to be confined to a certain area, in close vicinity of the monitoring system, thereby limiting the mobility of the patient. In addition, pinch pressure applied by a clip may overtime cause an uncomfortable feeling or become overbearing to the patient to the extent the patient may want to remove the sensor and cease otherwise required monitoring. As a result, such sensors are not suitable for the prolonged and continuous pulse oximetry measurements.

In recent development of wearable devices, wrist and smartwatches are using reflective optical sensors to estimate heart-rate. These devices are comfortable to use for long time, however their method of reflective optical sensors yields poor quality of PPG signals. Such a signal might be good enough to count a pulse, but the PPG quality is completely distorted, therefore making it useless for any attempt to analyze the wave form or to perform accurate pulse oximetry. The following publications show some known pulse and/or oximetry devices that are worn on the user's wrist: US2010/056934, US2009/247885, US2010/331709, US2002/188210, U.S. Pat. No. 6,210,340; JP2009160274, JP20052705443, JP2009254522, JP2010220939, JP2005040261; WO2010/111127; KR20110006990 GB2341233. Such known devices use either reflection (i.e. illuminate light at zero degrees) or transmission (illuminate light through the tissue).

WO2011/013132 describes a system for measuring one or more (light-absorption related) blood analyte concentration parameters, using a PPG device configured to effect a PPG measurement by illuminating the patient with at least two distinct wavelengths of light. This PPG device also determines relative absorbance at each of the wavelengths, with a dynamic light scattering measurement (DLS) device that is configured to effect a DLS measurement of the subject to rheologically measure a pulse parameter of the subject. The system also has electronic circuitry that is configured to temporally correlate the results of the PPG and DLS measurements in accordance with the temporal correlation between the PPG and DLS measurements, as well as assessing values of the light-absorption related blood analyte concentration parameters.

In a more recent patent application, US2014/0200423 describes a pulse oximeter placed on the distal end of the ulnar bone. This oximeter builds a dome over the styloid process to use its boney dome like shape as a reflector. This pulse oximetry device has a dome shaped structure arranged to fixate an area above a distal end of the ulna (or elbow bone), a detector positioned above the fixated area, and at least two light sources having different wavelengths located at a periphery of the fixated area. This detector is arranged to measure reflections by the distal end of the ulna of light emitted from the at least two light sources, with the reflections being at an angle in the range of 20-160° degrees from the emitted light.

It should be noted that none of the abovementioned publications describes a dedicated sensor for improved Signal to Noise Ratio (SNR) and analyzing the shape of the measured pulsating wave. It is therefore an object of the present subject matter to provide a system and method for estimating the spectrographic and hemodynamics of a subject circulatory system, based on the shape of the measured pulsating wave. Further objects and advantages will appear as the description proceeds.

### SUMMARY

According to a first aspect, a concave optical sensor capable of being in contact with the skin of a user is provided, the concave optical sensor comprising:

at least one light source having at least one wavelength, and configured to allow emission of light; and

at least one detector responsive to emitted light from the light source, wherein both the at least one light source and the at least one detector are positioned on a concave segment.

According to other embodiments, the angle between the at least one light source and the at least one light receiver is different from 0 or 180 degrees.

According to other embodiments, the concave optical sensor is activated when worn on boney structure or a body part of a user.

According to other embodiments, the boney structure or the body part are such that are not used for transmissive optical sensors.

According to other embodiments, the at least one light source comprises a single wavelength, and wherein a photoplethysmography (PPG) signal can be measured.

According to other embodiments, the at least one light source includes at least two wavelengths, and wherein pulse oximetry can be measured.

According to other embodiments, spectroscopic analysis of the blood constituents can be performed.

According to other embodiments, the sensor is coupled to an external device that is configured to allow calculation of pulse oximetry data.

According to other embodiments, the sensor is coupled to an external device that is configured to allow calculation of hemodynamic parameters by analyzing a pulse wave received in the at least one light receiver.

According to other embodiments, heart rate can be measured.

According to other embodiments, pulse oximetry can be measured.

According to other embodiments, a perfusion index can measured.

According to other embodiments, the sensor further comprising a display.

According to other embodiments, the sensor further comprising a communication module configured to allow wireless communication with external devices.

According to other embodiments, cardiovascular age of a user can be estimated.

According to other embodiments, the cardiovascular age can be used as a total health score.

According to other embodiments, the total health score is used as an assessment of life style wellness contribution.

According to other embodiments, the sensor further comprising a biometric module, wherein physiological signals from a user are used to validate the user identity.

According to other embodiments, the sensor is embedded within a flat segment.

According to other embodiments, the flat segment has a shape similar to a credit card.

According to other embodiments, the sensor is embedded within a computerized device.

According to other embodiments, the sensor is embedded within a steering wheel.

According to other embodiments, the sensor is embedded within a piece of clothing.

According to other embodiments, said piece of clothing is selected from a group of clothing such as a sock, a bra, a bathing suit, a shirt, pants, a combination therein or the like.

According to other embodiments, the sensor is embedded within weighing scales.

According to other embodiments, the sensor is embedded within sport training machine.

According to other embodiments, the sensor is embedded within a massaging device.

According to other embodiments, the sensor is embedded within an electrocardiogram (ECG) patch.

According to other embodiments, the sensor is embedded within a patch that can temporarily be attached to a user's body.

According to other embodiments, the sensor is water proof.

According to a second aspect, a monitoring device is provided that comprises:
at least one concave optical sensor; and
a microcontroller that is clipped over a wearable device configured to at least partially wrap about a body part of a user.

According to other embodiments, the microcontroller is clipped on a strap over the body part.

According to other embodiments, the microcontroller is strapped about a wrist or a finger of the user.

According to other embodiments, the at least one concave sensor acts as a pulse oximeter.

According to a third aspect, a monitoring system is provided that comprises:
a concave optical sensor;
a wearable device coupled to a concave optical sensor;
a communication module configured to allow wireless transmission of data; and
a computing device configured to allow receiving signals from the communication module so as to perform a pulse wave analysis and derive hemodynamic parameters.

According to other embodiments, said wearable device is a ring, a watch, a wrist band, a combination thereof and the like.

In accordance with a forth aspect, a signal processing method is provided that comprises:
providing at least one photoplethysmography (PPG) sensor;
providing at least two synchronized channels; and
employing the at least one PPG sensor to use the synchronized channels so as to get a combined pulse shape with reduced noise.

In accordance with a fifth aspect, a signal processing method is provided that comprises:
providing an ECG wave signal;
triggering at least one PPG signal with the ECG wave signal so as to form an average pulse with reduced noise.

According to other embodiments, the method further comprising identifying real pulses using wavelets and high degree derivatives.

In accordance with a sixth aspect, an elastic concave optical sensor is provided that comprises:
at least one light source having at least one wavelength, wherein the light source is configured to emit light beams; and
at least one light receiver responsive to the light beams emitted from the light source,
wherein both the at least one light source and the at least one light receiver are positioned on an elastic concave segment that is pressing the concave sensor against a body part of a user so as to obtain optimal coupling.

According to other embodiments, the elastic concave segment stabilizes the sensor against the body part to prevent motion artifacts.

According to other embodiments, the sensor further provided with a resilient element configured to press the sensor against a body part.

According to other embodiments, said resilient element is a spring or a spring like element.

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIG. 1 schematically illustrates commercially available sensors and methods for measuring pulse oximetry from a finger.

FIG. 2 schematically illustrates a cross-sectional view of a concave optical sensor positioned over the finger of a user, according to an exemplary embodiment.

FIG. 3A schematically illustrates a cross sectional view of a ring shaped housing placed on a finger, according to an exemplary embodiment.

FIG. 3B schematically illustrates a perspective view of a ring shaped housing, according to another exemplary embodiment.

FIG. 3C schematically illustrates a perspective view of a ring shaped housing, according to yet another exemplary embodiment.

FIG. 3D schematically illustrates a perspective view of a ring shaped housing, according to another exemplary embodiment.

FIG. 4A shows an image of the concave optical sensor coupled with a dedicated wearable device capable of displaying the pulse and oxygen levels on a display, wherein the user places a finger onto the concave optical sensor, according to an exemplary embodiment.

FIG. 4B shows an image of the concave optical sensor coupled with a dedicated wearable device capable of displaying the pulse and oxygen levels on a display, wherein the user places a finger into the concave optical sensor, according to an exemplary embodiment.

FIG. 4C shows an additional image of the concave optical sensor coupled with a dedicated wearable device capable of displaying the pulse and oxygen levels on a display, wherein the user places a finger onto the concave optical sensor, according to an exemplary embodiment.

FIG. 4D shows a further image of the concave optical sensor coupled with a dedicated wearable device capable of displaying the pulse and oxygen levels on a display, wherein the user places a finger onto the concave optical sensor, according to an exemplary embodiment.

FIG. 5A schematically illustrates a perspective view of a concave optical sensor in a wrist housing, according to an exemplary embodiment.

FIG. 5B schematically illustrates an enlarged segment of the wrist housing, according to an exemplary embodiment.

FIG. 6 shows an image of concave optical sensors embedded into the sides of a computerized device, according to an exemplary embodiment.

FIG. 7 schematically illustrates a steering wheel 80 with an embedded concave optical sensor, according to an exemplary embodiment.

FIG. 8 schematically illustrates a piece of clothing with an embedded concave optical sensor, according to an exemplary embodiment.

FIG. 9A schematically illustrates a concave optical sensor attached to the body of a user, according to an exemplary embodiment.

FIG. 9B schematically illustrates a concave optical sensor monitoring the body of a baby, according to an exemplary embodiment.

FIG. 10 schematically illustrates weighing scales with an embedded concave optical sensor, according to an exemplary embodiment.

FIG. 11 schematically illustrates sport training machine with an embedded concave optical sensor, according to an exemplary embodiment.

FIG. 12 schematically illustrate schematically illustrates a concave optical sensor coupled to the body of the user, according to an exemplary embodiment.

FIG. 13A schematically illustrates a side view of a flat pulse oximeter with a concave optical sensor that is gripped by a finger of the user, according to an exemplary embodiment.

FIG. 13B schematically illustrates a perspective view of the flat pulse oximeter, according to an exemplary embodiment.

FIG. 14 schematically illustrates a concave optical sensor embedded into a clinical thermometer, according to an exemplary embodiment.

FIG. 15 schematically illustrates a concave optical sensor embedded into a bra, according to an exemplary embodiment.

FIG. 16 schematically illustrates, according to an exemplary embodiment, a concave optical sensor integrated with a ring.

FIG. 17 schematically illustrates, according to an exemplary embodiment, a concave optical sensor integrated with a wrist watch, further comprising an elastic member.

FIG. 18 schematically illustrates, according to an exemplary embodiment, a concave optical sensor integrated with a wrist bracelet, further comprising an elastic member and a force limiter.

FIG. 19 schematically illustrates, according to an exemplary embodiment, a concave optical sensor integrated with an earbud, further comprising an elastic member.

FIG. 20 schematically illustrates, according to an exemplary embodiment, a concave optical sensor integrated with a device that is normally held by a user's palm, for example a portable cell phone, also known as a mobile phone, further comprising an elastic member

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

For clarity, non-essential elements were omitted from some of the drawings.

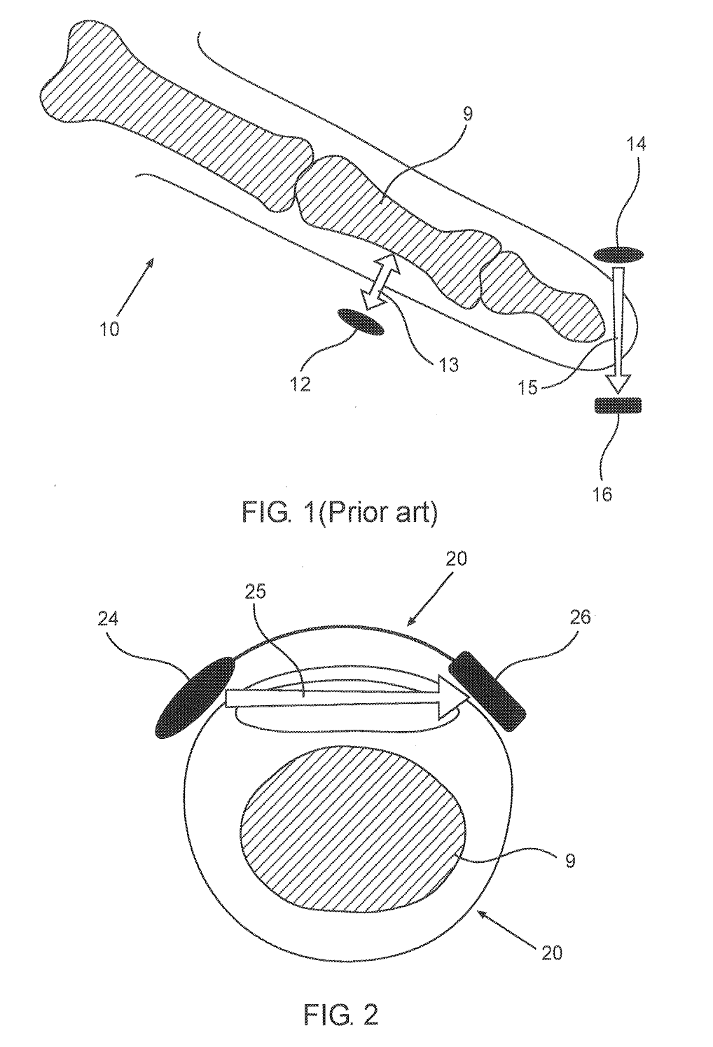

FIG. 1 schematically illustrates commercially available sensors and methods for measuring pulse oximetry from a finger 10. A first method for measuring pulse oximetry from the finger 10 incorporates the use of a reflective optical sensor 12, comprising a light source and a light receiver. The light source is configured to send a reflected optical beam 13 towards a tissue surrounding the bone 9 of the finger 10. It should be noted that with the reflective optical sensor 12, both the light source and the light receiver are on the same pane having zero degrees between them.

The beam 13 is then reflected from the bone 9 and returned to the light receiver of the reflective optical sensor 12 such that a pulse is measured. It is appreciated that the reflective method yields poor quality of photoplethysmography (PPG) signals, mainly due to the phenomenon that about 95% of the reflected beam 13 is reflected from the surface of the tissue, e.g. the skin, instead of being used for the measurement (e.g. as a result of noise). It should also be noted that any light reflected back from the surface causes energy loss and therefore reducing the efficiency of the measurement.

A second method for measuring pulse oximetry from the finger 10 incorporates the use of a transmissive optical light sensor, comprising a light source 14 and a light receiver 16 opposite to each other, usually provided as a clip (or “crocodile” type) light sensor that illuminate light 15, through the tissue, from one side of a boneless tissue to the other side. It is appreciated that wearing such a device for long periods of time is uncomfortable to the user, due to the positioning on the fingertip.

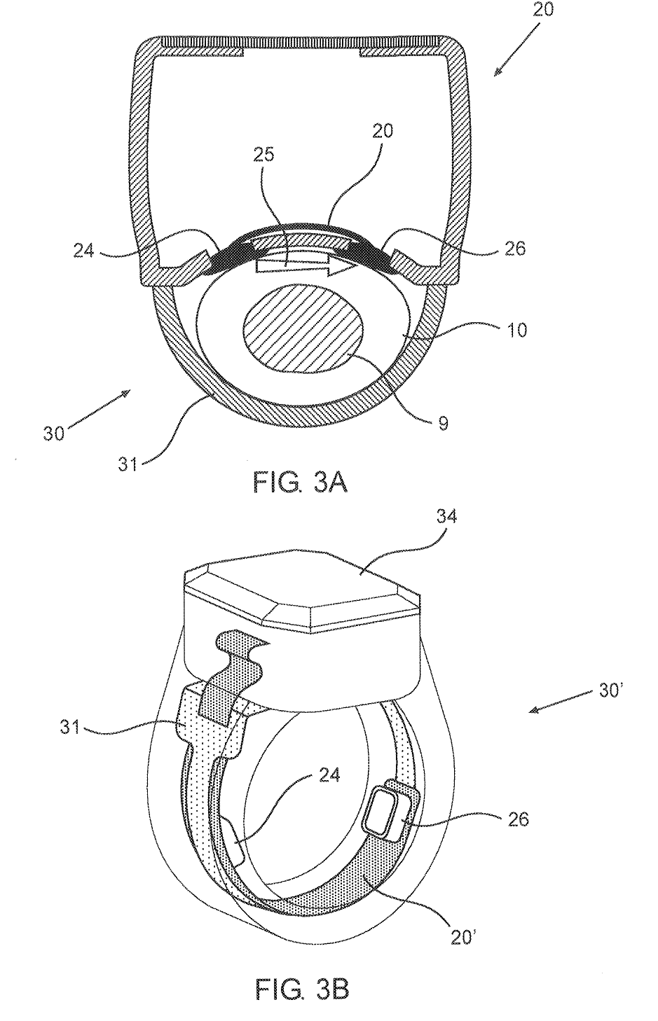

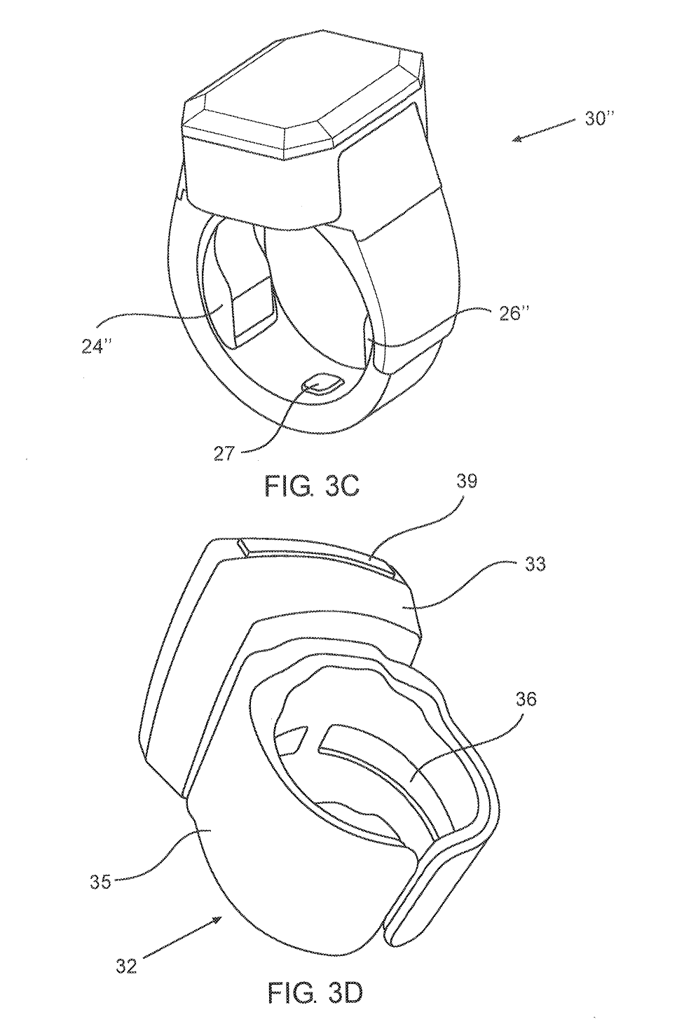

Referring now to FIGS. 2 and 3A-3D, these figures show an improved solution for measuring pulse oximetry using a concave optical sensor. FIG. 2 schematically illustrates a cross-sectional view of a concave optical sensor 20 positioned over the finger 10 of a user. The concave optical sensor 20 comprises at least one light source 24, typically a light emitting diode (LED), and at least one light receiver 26, typically a photodiode. The at least one light source 24 and the at least one light receiver 26 are positioned in an angle one relative to the other. For optimal operation, the cross-section of the finger 10 may be regarded as a clock, and then the concave optical sensor 20 should be placed on hours seven and five, or eight and ten, or eleven and one, etc.

The at least one light receiver 26 is configured to measure a light beam 25 emitted from the light source 24 and passing through the tissue of the finger 10, whereby due to the concave shape of the concave optical sensor 20 an improved measurement may be achieved. Specifically, due to the concave shape, the measurements with the at least one light source 24 and the at least one light receiver 26 at 0° in between (i.e. the reflective method) and/or 180° in between (i.e. the transmissive method) are excluded from consideration.

In some embodiments, the concave optical sensor 20 includes a pair of red and Infra-Red (IR) LEDs as the light source 24, together with a Photodiode as the light sensor 26 positioned on the other side of the finger. It should be noted that unlike the tip of the finger (where there is no bone) the finger root includes a bone and therefore the LED-PD pair needs to be located in a circular arc, which is attached to the finger. Optionally, the light source 24 uses beams with different wavelengths.

It is appreciated that in contrast to the commercially available solutions, the concave optical sensor may be worn on a boney part of the finger while transferring the beam in the transmissive method through the tissue (without reflections from the bone). Thus, a high quality measurement may be achieved in places that normally are considered unsuitable for such measurements. Additionally, the user may wear the concave optical sensor for long periods of time, for instance as a ring, without feeling any discomforts (in contrast to the crocodile clips) and without interfering with normal functioning of the hand and fingers.

Furthermore, the concave optical sensor provides a better signal to noise ratio, if a finger is placed on it, compared to putting the finger on a flat surface and using a reflective pulse oximetry sensor (as suggested by the commercially available solutions).

FIG. 3A schematically illustrates a cross sectional view of ring shaped housings 30 worn on finger 10 of a user, wherein a bone 9 can be seen. The ring shaped housing 30 may be configured to comprise a concave optical sensor 20 with light source 24 and the light sensor 26. The direction of the light beam 25 is shown.

FIG. 3B schematically illustrates a cross-sectional view of a ring shaped housing 30′ comprising a concave optical sensor 20′. The ring shaped housing 30′ is configured to fit onto a finger of a user, such that a strap 31 that can optionally be a metal support, envelops the finger 10. The strap can comprise a microcontroller. The concave optical sensor 20 may be embedded into the ring shaped housing 30′ such that the light beam may be emitted and received by the concave optical sensor 20′, while passing through the tissue of the finger wearing the ring. Preferably, the light beam is emitted from the light source 24 and received by the light receiver 26.

The ring shaped housing 30′ also comprises a processor unit capable of processing the information measured by the sensors, and also a display 39 configured to display information to the user. For example, the display 39 continuously displaying the pulse and oxygen levels.

Optionally, the concave optical sensor may be positioned proximally to the processor unit and the display.

FIG. 3C schematically illustrates a perspective view of another embodiment of a ring shaped housing 30″. The light receiver element in the concave sensor in the ring is replaced by a component that includes both the light receiver and an analog front end chip 24″ with the following capabilities:

a. It can generate PPG signal when the finger is placed inside the ring by using the light receiver and circuit for controlling the LEDs 26″ on the other side of the ring. The LEDs can be of different wave lengths or s range of wave length that can be received by the light sensor.

b. The Chip includes also the analog front end for electrical signals for ECG. For this purpose the ring has at least one dry electrode 27 inside the ring and one electrode to be touched by the contralateral hand or leg.

c. The Chip includes high frequency generators that can inject these frequencies to the body to measure bio-impedance. The electrical frequency delivered through at least 2 electrodes is in frequency of several kilohertz. Optionally, 2 or 4 electrodes in Kelvin arrangement can be utilized, where 2 electrodes are used to inject the frequencies and 2 for receiving.

By using 2-4 internal electrodes, one can get an impedance plethysmograph (IPG) that acts similar to the PhotoPlethysmoGraph (PPG) discussed herein before. By using at least one and preferably 2 internal electrodes and one but preferably 2 external electrodes (to be touched by contralateral hand or leg) we can perform body composition measurement of percentage of body fat, body muscles, water, etc.

The electrodes and analog front end can be used for measuring Gslvsnic Skin Response (GSR). In this way, the ring can perform full set of electrical and optical measurement of many parameters of the physiology of the user body with one small size form wearable like a ring, wrist band or earphone.

FIG. 3D shows a perspective view of an additional ring shaped housing 32. It is appreciated that the additional ring shaped housing 32 has two main portions: a top portion 33 comprising the display 39, and a bottom portion 35. Preferably, the top portion 33 further comprises various processing, computational and controlling elements that are required for analyzing the measured signal.

In some embodiments with wireless communication to a corresponding computerized device (e.g. mobile phone or tablet), a display may not be necessary due to the communication with the corresponding device that provides the display.

The bottom portion 35 may be elastic so as to fit onto different portions of the body (e.g. fit onto fingers of different size). The bottom portion 35 may also comprise an inner segment 36 (e.g. having an arc like shape) with various sensors in order to allow the measurement of the required medical information. It is appreciated that the elastic finger attachment of the ring shaped housing ensures that the sensors are pressed against the skin, and the pressure is in the correct range. Too little pressure might not provide good coupling while too much pressure may squeeze the blood out of the pressed tissue and might result in low Perfusion Index (ratio between the pulsatile part and the constant part).

In some embodiments, the ring shaped housing may further comprise a communication module capable of wirelessly transmitting information to an external device, for instance to a smartphone or PC. In a preferred embodiment, the ring shaped housing may further comprise a power storage unit or battery.

It should be noted that the structure of the ring shaped housing has precise positioning of the components of the optical sensor (i.e. the LED and the light receiver like Photodiode or Light to Frequency converter), such that the emitted light beam is not blocked by the bone. Additionally, the precise positioning ensures that the reflective method is not used, whereby the majority of the light beam is reflected from the outer surface of the skin and not from the capillary bed. Furthermore, the ring shaped housing has miniaturized electrical components that are required due to the small size of a finger.

As shown herein before, the sensor can be embedded within a wearable device that wrappes about a body part such as a finger or a wrist, however, it should be understood that it can also be partially wrapped about the body part.

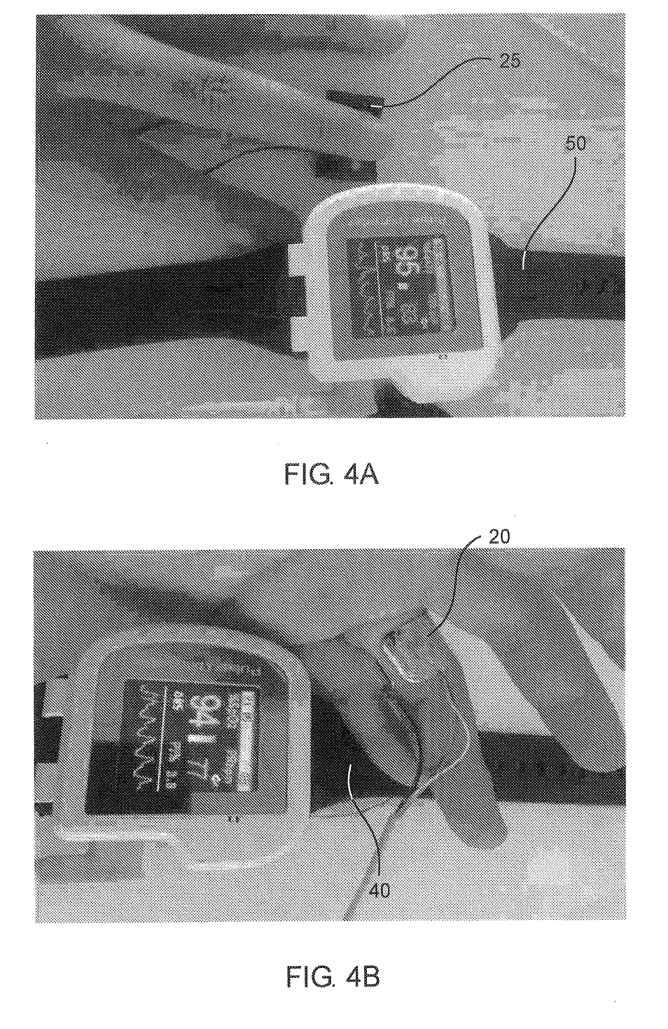

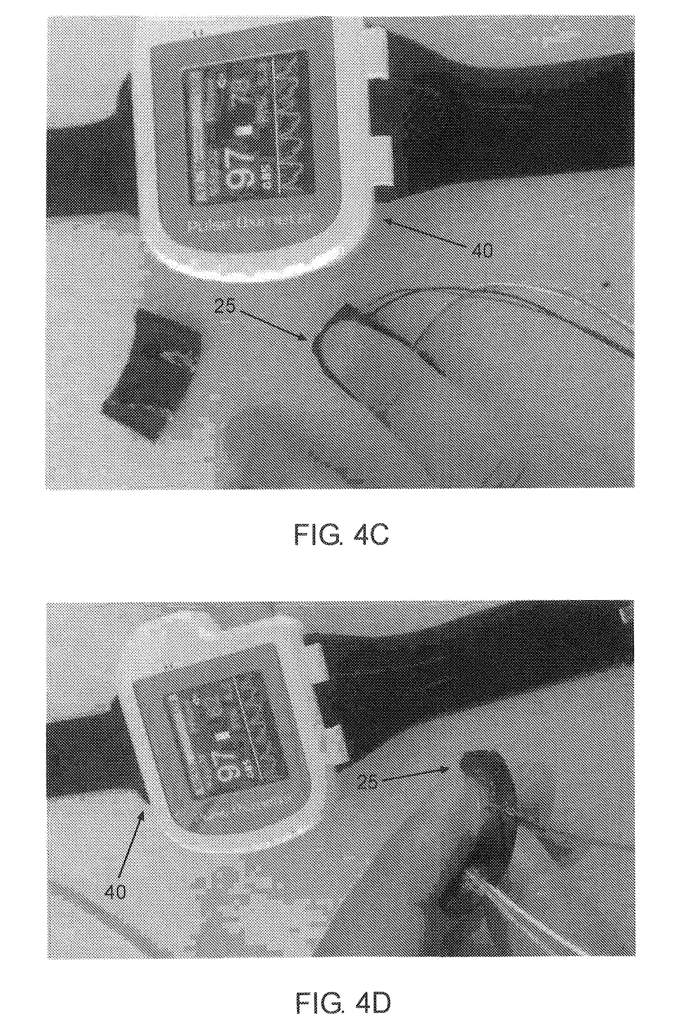

Referring now to FIGS. 4A-4D, these figures show images of the concave optical sensor and wearable devices.

FIG. 4A schematically illustrates a concave optical sensor 25 coupled with a dedicated wearable device 40 capable of displaying the pulse and oxygen levels on a display, wherein the user places a finger onto the concave optical sensor 25. In some embodiments, it is possible to modify existing pulse oximeters (e.g. clip type sensors) with the concave optical sensor such that the user is only required to place a finger onto the concave optical sensor 25 (operating only by touch) instead of inserting the finger.

FIG. 4B shows a concave optical sensor 20 coupled with a dedicated wearable device 40 capable of displaying the pulse and oxygen levels on a display, wherein the user places a finger into the concave optical sensor 20.

It should be noted that the concave optical sensor may be provided as a bare sensor, having only a light source (e.g. LED) and a light receiver (e.g. photodiode) on a substrate, such that the user only presses the skin onto that substrate in order to allow monitoring. Optionally, that substrate may be coupled to a processor (e.g. a processing chip) configured to allow analysis with at least one of the following:
a pulse oximetry algorithm;
a blood pressure algorithm; and
estimation of the total health score that is expressed as physiological age.

FIGS. 4C and 4D shows a concave optical sensor 25 coupled with a dedicated wearable device 40 capable of displaying the pulse and oxygen levels on a display, wherein the user places a finger onto the concave optical sensor 25.

It is appreciated that the ring pulse oximeter is a new generation of pulse oximeters that can be worn for a longer period of time without being obtrusive. The ring pulse oximeter measures oxygen saturation in the root of the finger instead of the fingertip, and therefore it is more stable, fits the finger tightly and is less susceptible to motion artifacts and to poor blood circulation due to cold weather. Users that are interested in getting to know their oxygen saturation changes during exercise, active life style and hiking in the mountains, airplane flights and other recreational activities may greatly benefit from this high-quality pulse oximeter (compared to old generation crocodile type devices).

It should be noted that while the abovementioned solution teaches placing the concave optical sensor onto a finger, the same may apply to other parts of the body. For instance, a wearable wrist device (e.g. similar to a smartwatch) that receives pulse oximeter signals from the wrist. Another example is wearable earphones that can provide longer periods of monitoring.

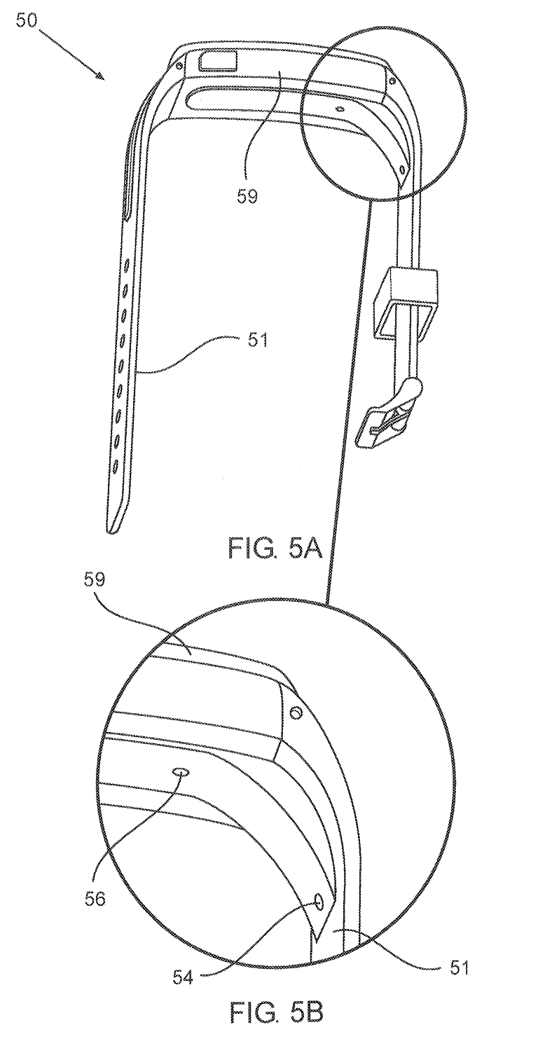

Referring now to FIGS. 5A-5B, these figures show an improved solution for measuring pulse oximetry using a concave optical sensor that is wearable on a wrist of the user. FIG. 5A schematically illustrates a perspective view of a concave optical sensor in a wrist housing 50. The wrist housing 50 comprises a band 51 capable of surrounding the wrist of the user (similarly to the strap of a watch). The wrist housing 50 also comprises a display 59 (similarly to the ring housing, shown in FIGS. 3A-3B), that is configured to display information to the user.

In some embodiments, the wrist housing may further comprise a communication module capable of wirelessly transmitting information to an external device, for instance to a smartphone or PC. In a preferred embodiment, the wrist housing may further comprise a power storage unit or battery.

FIG. 5B schematically illustrates an enlarged segment of the wrist housing 50. The wrist housing 50 comprises the concave optical sensor at one side of the band 51 (in contrast to the ring housing, with the concave optical sensor positioned at the center). The concave optical sensor of the wrist housing 50 therefore comprises a light source 54 and a corresponding light receiver 56, such that light may be emitted from the light source 54 towards the light receiver 56 and pass through the tissue of the wrist of the user.

Optionally, a concave optical sensor may be embedded into the band of an existing wearable device (such as a watch or a smartwatch), and then operate similarly to the wrist housing 50 with data collected from the concave optical sensor.

In some embodiments, a similar device may be fitted onto other parts of the human body. For example, a wearable band that surrounds a leg of a user.

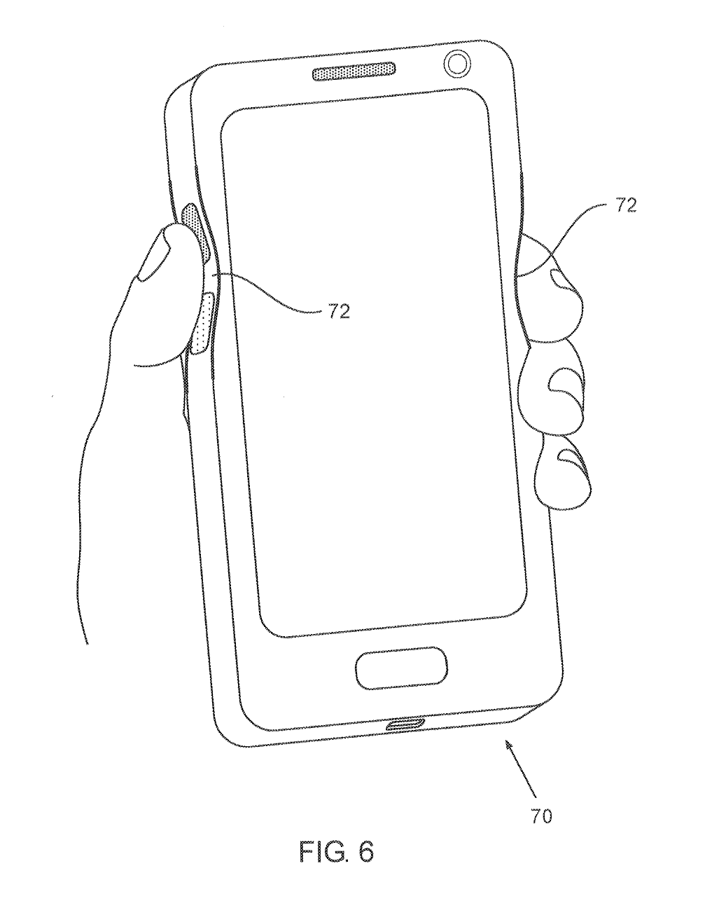

Referring now to FIG. 6 schematically illustrates concave optical sensors 72 embedded into the sides of a computerized device 70 (e.g. a smart phone or tablet). By embedding the concave optical sensors 72 to items that are held by the user on a daily basis, it may be possible to measure the required signal such that the user is not required to change the daily routine. Optionally, the concave optical sensors 72 embedded into the sides of a computerized device 70 may be coupled with the processor of the computerized device so as to display the information on the built-in display, or alternatively may be coupled to an external device to store the measured data.

Optionally, the sensor can be embedded within an elastic cushion (not shown in this figure).

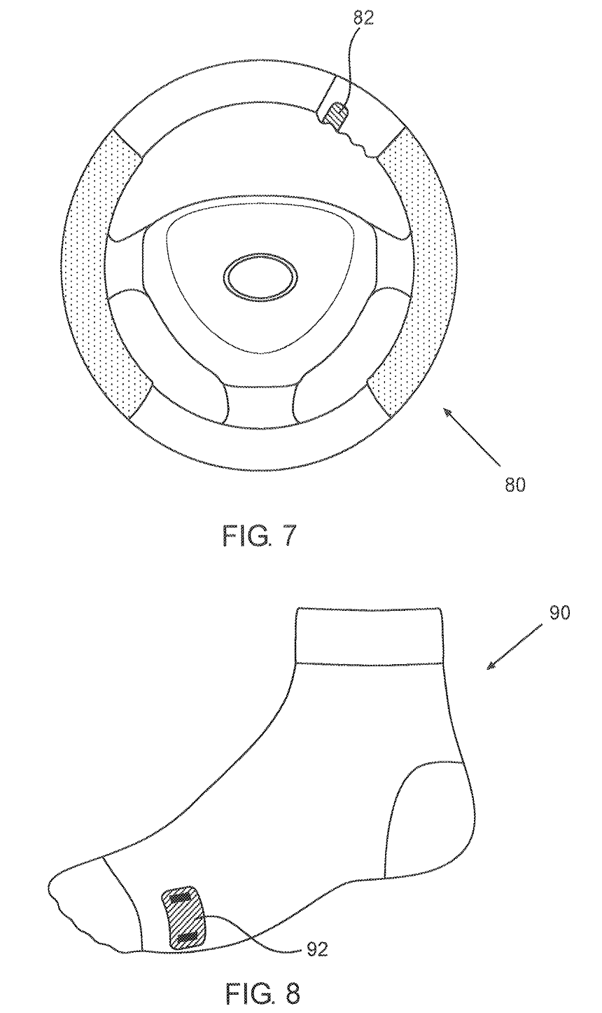

Referring now to FIG. 7, schematically illustrates a steering wheel 80 with an embedded concave optical sensor 82. In some embodiments, a concave optical sensor may be embedded to other daily use objects in order to monitor cardiovascular activity. For instance, an light source and a corresponding light receiver (as the concave optical sensor 82) embedded into a steering wheel 80 of a car, such that unobtrusive tracking of a driver's health condition may be achieved. Practically, a monitor device is formed.

Referring now to FIG. 8, schematically illustrates a piece of clothing 90 with an embedded concave optical sensor 92. In some embodiments, the concave optical sensor 92 may be embedded into fabrics that are worn by the user. Thus, the concave optical sensor 92 may be embedded into clothes and/or shoes so as to allow continuous monitoring. For instance, a sock 90 with an embedded concave optical sensor 92 may provide continuous monitoring with the user's skin (of the foot) constantly in contact with the embedded concave optical sensor 92 as long as the sock is worn by the user.

Such concave optical sensors 92 embedded in a sock 90, may be particularly useful for monitoring infants, monitoring during a sport activity, or for regular continuous monitoring of patients in hospitals or at home (in contrast to current methods of obtrusive detectors).

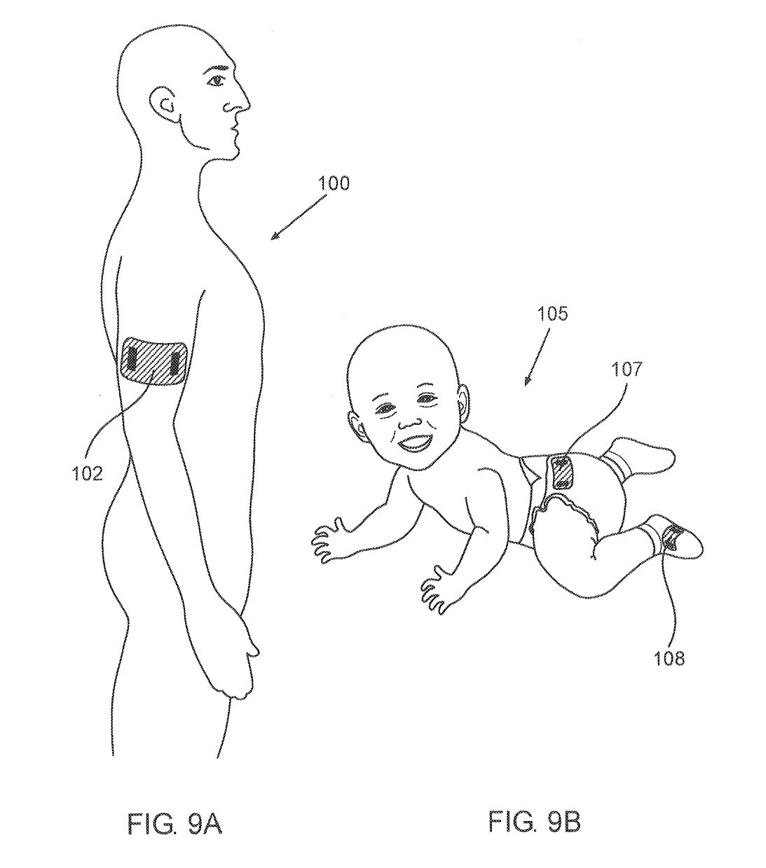

Referring now to FIG. 9A, schematically illustrates a concave optical sensor 102 attached to the body of a user 100. In some embodiments, the concave optical sensor 102 may be provided as a patch that is temporarily attached to the body of the user 100 with an adhesive, or alternatively attached with a dedicated strap. Such a patch may allow continuous monitoring of cardiovascular health signals of the user 100. For example, the patch may be attached to the arm of the user 100, and a light beam is emitted from a light source of the concave optical sensor 102, passes through the tissue of the arm, and is then received by a light receiver of the concave optical sensor 102.

Referring now to FIG. 9B, this figure schematically illustrate a concave optical sensor 107, 108 monitoring the body of a baby 105. It should be noted that commercially available devices for infant monitoring typically use transmissive pulse oximeters due to the tiny feet of babies. Therefore, using the abovementioned sock or patch with the concave sensors may provide a convenient solution for monitoring babies. Specifically, the sock worn by the baby or alternatively attaching a patch 108 to the feet of the baby 105. Optionally or alternatively, a concave optical sensor may also be embedded in a baby's diapers clip 107. In each case, the concave optical sensor is in contact with the skin of the baby 105, whereby the light passes from the source to the receiver through the tissue of the baby 105.

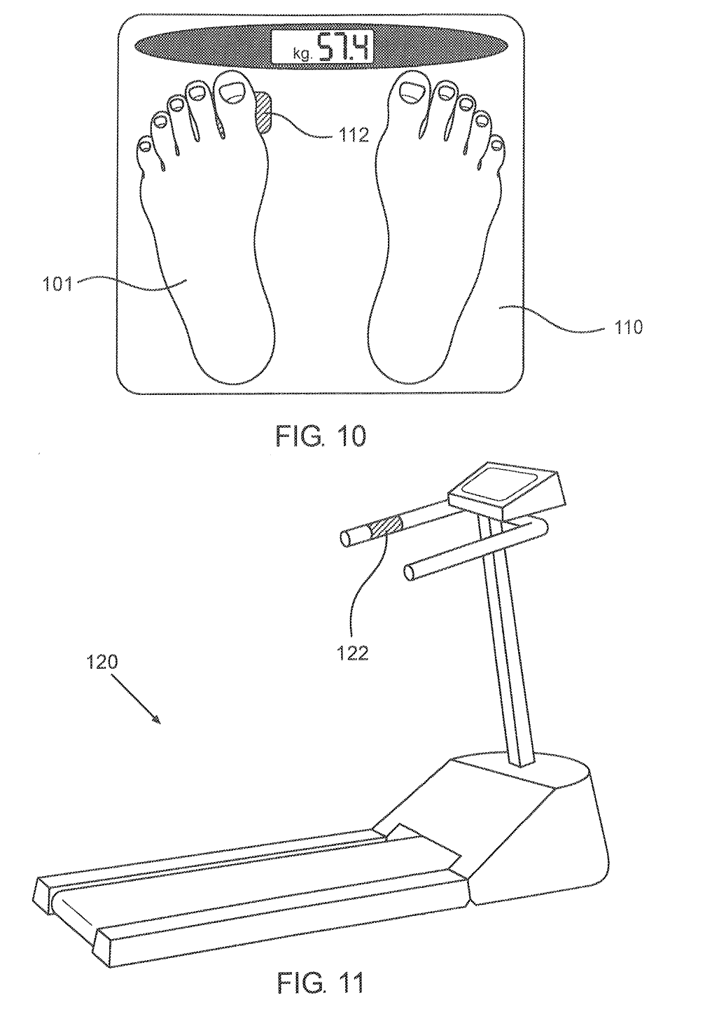

Referring now to FIG. 10, schematically illustrates weighing scales 110 with an embedded concave optical sensor 112. In some embodiments, a concave optical sensor 112 may be embedded into weighing scales 110 that are typically found in use for measuring the weight of users (by usually standing on the scales). For instance, the concave optical sensor 112 may be embedded into a “bathroom scale” so that a user stepping barefoot onto the scales automatically initiates the cardiovascular monitoring, whereby the skin of the user contacts the embedded concave optical sensor 112. For example, the user placing a foot 101 onto the weighing scales 110, with a light beam emitted from a light source of the concave optical sensor 112, passing through the tissue of the foot, and then received by a light receiver of the concave optical sensor 112. Optionally, a pressure sensor may initiate the operation of the concave optical sensor 112 if a predetermined minimal weight is detected.

Referring now to FIG. 11, this figure schematically illustrates a sport training machine 120 with an embedded concave optical sensor 122. In some embodiments, a concave optical sensor 122 may be embedded into sport training machines 120, such as can be found in a gym. For instance, the concave optical sensor 122 may be embedded into a part of the machine 120 to be gripped by the hands of the user, similarly to current commercially available ECG electrodes that are embedded into such machines.

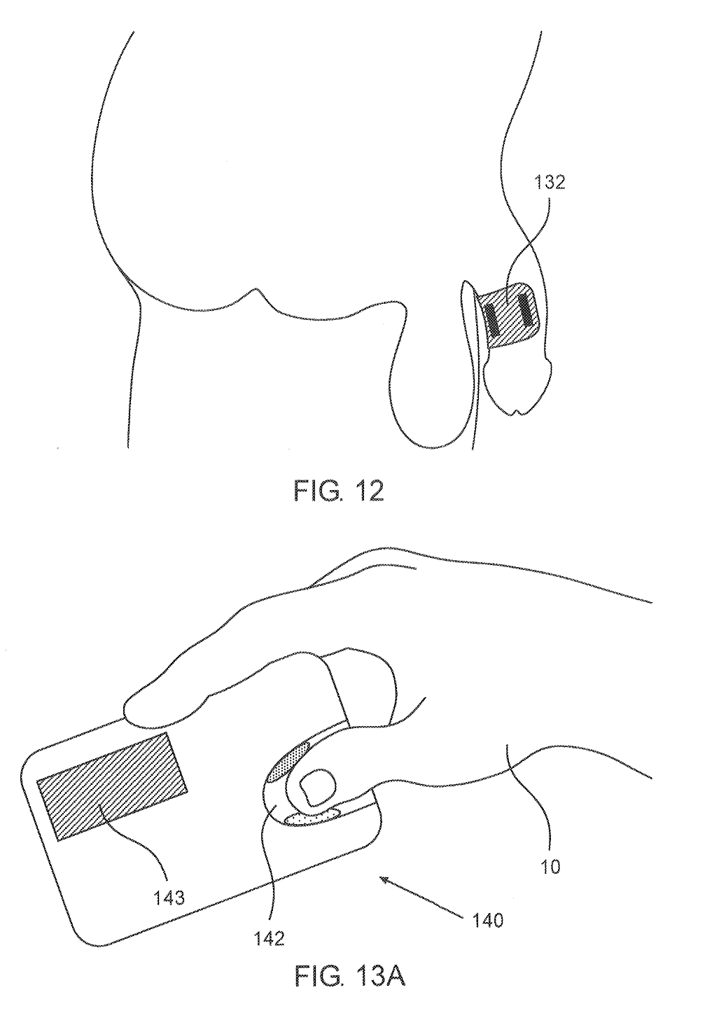

Referring now to FIG. 12, schematically illustrates a concave optical sensor 132 coupled to the body of the user. The concave optical sensor 132 may be coupled to the body of the user for instance in order to allow estimation of improvement in cardiovascular operation. For example, the concave optical sensor 132 may be coupled to male genitals in order to monitor operation of the cardiovascular system with a light beam emitted from a light source of the concave optical sensor 132, passing through the tissue of the body, and then received by a light receiver of the concave optical sensor 132.

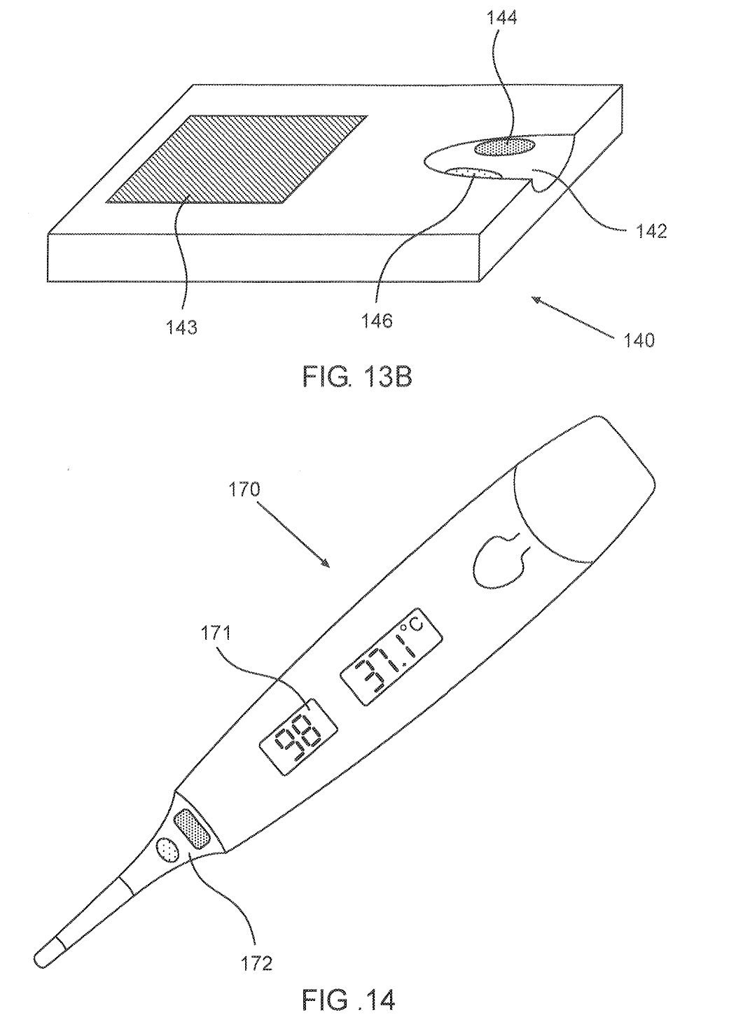

Referring now to FIGS. 13A-14B, these figures show a flat pulse oximeter. FIG. 13A schematically illustrates a side view of a flat pulse oximeter 140 with a concave optical sensor 142 that is gripped by a finger 10 of the user, and FIG. 13B schematically illustrates a perspective view of the flat pulse oximeter 140.

In some embodiments, the concave optical sensor 142 may be embedded into a flat substrate such that the user only places a finger 10 onto the concave optical sensor 142 so as to allow measurement of the desired data, instead of wrapping the sensor around a portion of the body (e.g. around the finger). It should be noted that such usage of the sensor is similar to the concave optical sensors shown in FIGS. 4A, 4C, and 4D, as it replaces the clip type (“crocodile”) sensors used today.

It is appreciated that by providing the flat pulse oximeter 140 in small sizes (for instance in a credit card size), the flat oximeter 140 may be constantly carried by the user (e.g. in a wallet) such that it may be used at any desired moment. Preferably, the flat pulse oximeter 140 has an elliptical indentation in which the concave optical sensor 142 may be embedded, with a light source 144 (e.g. LEDs) and a corresponding light receiver 146 (e.g. photodiode). Optionally, the light source 144 and light receiver 146 are positioned on an elastic foil in the indentation for convenient pressing of the finger 10. Thus, the user may simply place the finger 10 in the indentation in order to initiate the measurements. Furthermore, such a small and light flat pulse oximeter 140 is easily carried in a user's pocket and does not have mechanical moving parts that can break thereby preventing wear.

In some embodiments, the flat pulse oximeter 140 may also comprise a display 143, for instance an e-paper display that is suitable for small devices and particularly for credit card sizes. Preferably, the display 143 shows oximetry and pulse data.

It should be noted that optimal operation of the concave optical sensors requires the contact of the sensors with slight pressure of the sensors to skin in order to get good signal, while no contact or too high pressure may cause big deterioration in signal quality and signal to noise ratio (SNR).

Referring now to FIG. 14, schematically illustrates a concave optical sensor 172 embedded into a clinical thermometer 170. The concave optical sensor 172 may be embedded to the thermometer 170 in a portion that contacts the skin of the user so as to allow the pulse oximetry measurement. Optionally, the results of the pulse oximetry measurement may be displayed in a dedicated display 171.

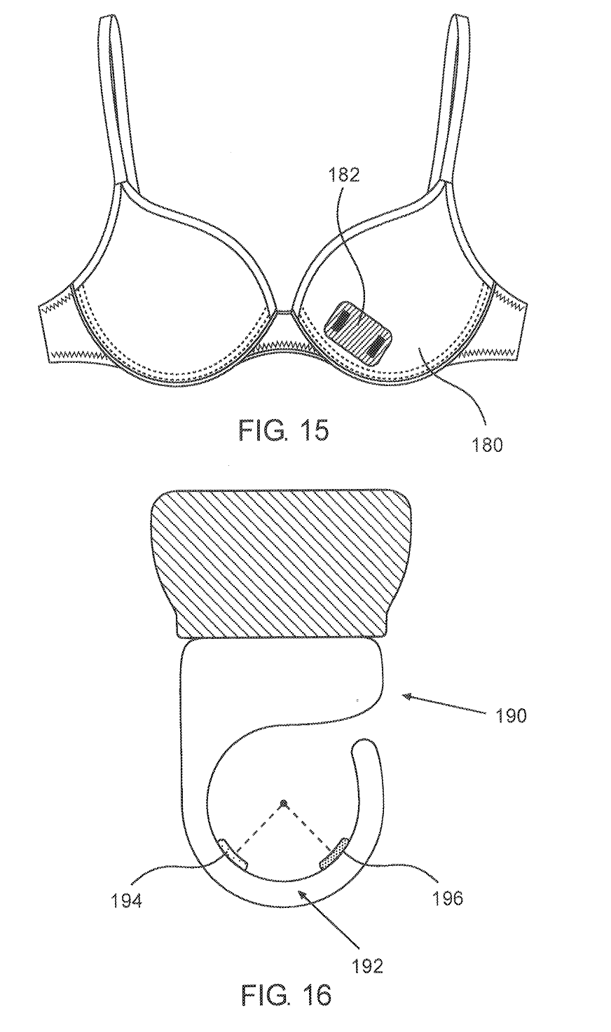

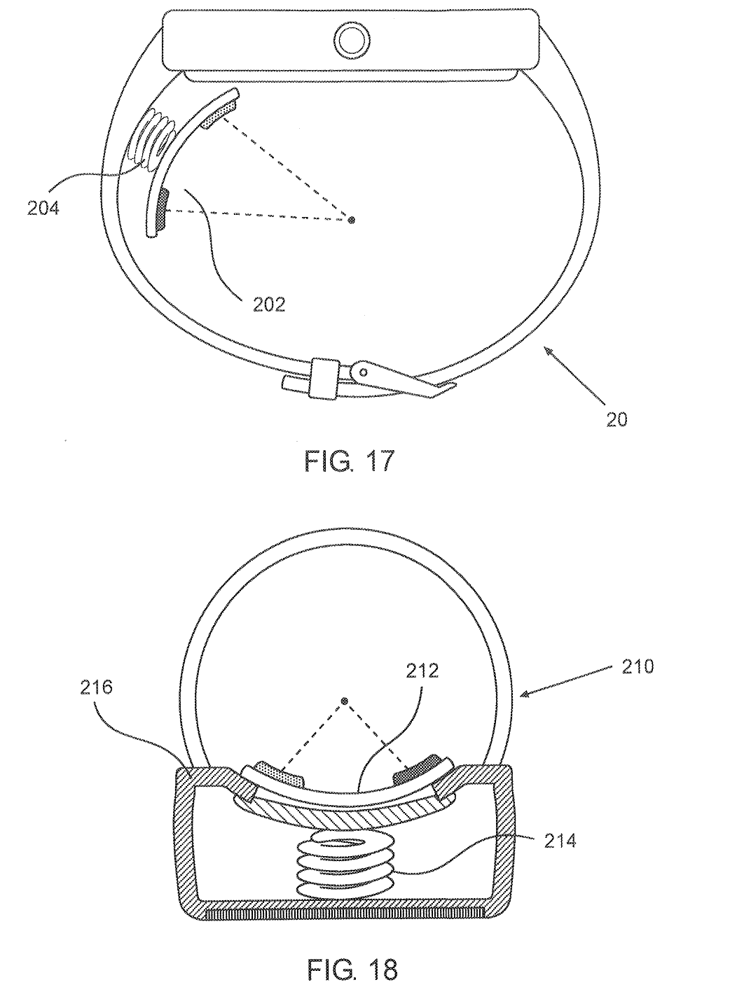

Referring now to FIG. 15, schematically illustrates a concave optical sensor 182 embedded into a bra 180. This daily use piece of clothing embedded with the concave optical sensor 182 may provide means for continuous monitoring. According to some embodiment, the concave optical sensor 182 is water proof may be embedded into a bra of a swimsuit. This is especially suitable for people that often visit hot springs and/or take hot bath every night as a way to increase circulation. Thus, a water proof patch or clip may give an indication of how long to stay in the water as it reduces blood pressure, and might have negative effect if people stay too long in high temperature.

Generally, the sensor depicted herein can be embedded to within any clothing of choice such as, but not limited to a sock, a bra, a bathing suit, a shirt, pants, a combination therein or the like. The sensor can be embedded within any equipment or medical devices such as but not limited to a massaging device, sport activity devices, an electrocardiogram (ECG) patch or any other medical device.

A measurement with an optical sensor in general, and with the concave optical sensor disclosed herein in particular, depends on the force of contact between the concave optical sensor and the skin, which is a function of the pressure exerted by the concave optical sensor on the skin. Too low force of contact may result in poor coupling so not enough light penetrates the capillary bed. On the other hand, a too high force of contact may squeeze blood out of the measured tissue and may result in poor signal quality. Furthermore, a wearable apparatus comprising an optical sensor is subject to movements of the user that may influence the contact force of the optical sensor against the user's skin. Therefore there is a need to keep the pressure of the optical sensor on the skin constant and in a certain force range.

According to some embodiments the present subject matter provides a concave optical sensor comprising an elastic or resilient member, for example a spring, configured to hold the concave optical sensor pressed against the skin constantly and in a certain contact force. This is achieved by using an elastic member having a spring constant k that is suitable to press the concave optical sensor against the skin in a suitable pressure, as discussed above.

FIG. 16 schematically illustrates, according to an exemplary embodiment, a concave optical sensor 192 integrated with a ring 190. According this embodiment, the ring 190 structure serves as an elastic member that presses the concave optical sensor against the skin constantly and in a certain contact force. The LED 194 and the PD 196 comprises the sensor.

FIG. 170 schematically illustrates, according to an exemplary embodiment, a concave optical sensor 202 integrated with a wrist watch 200, further comprising an elastic member 204, for example a spring, positioned adjacent to the concave optical sensor 202 in a manner that presses the concave optical sensor 202 against the skin constantly and in a certain contact force.

FIG. 18 schematically illustrates, according to an exemplary embodiment, a concave optical sensor 212 integrated with a wrist bracelet 210, further comprising an elastic member 214 and a force limiter 216. According to some embodiments, the elastic member 214 is a spring, positioned adjacent to the concave optical sensor 212 in a manner that presses the concave optical sensor 212 against the skin constantly and in a certain contact force. According to additional embodiment, the force limiter 216 is positioned adjacent to the concave optical sensor 212 in a manner that limits the force exerted by the elastic member 214 on the concave optical sensor. Thus, the force limiter 216 is configured to additionally control the contact force exerted by the concave optical sensor 212 against the skin, and aids in adjusting the contact force more accurately. It should be noted that inclusion of the force limiter 216 is not limited to the concave optical sensor 212 embedded in a wrist bracelet 210 and further comprising an elastic element, but rather any type of a concave optical sensor comprising an elastic element and a force limiter is under the scope of the present subject matter.

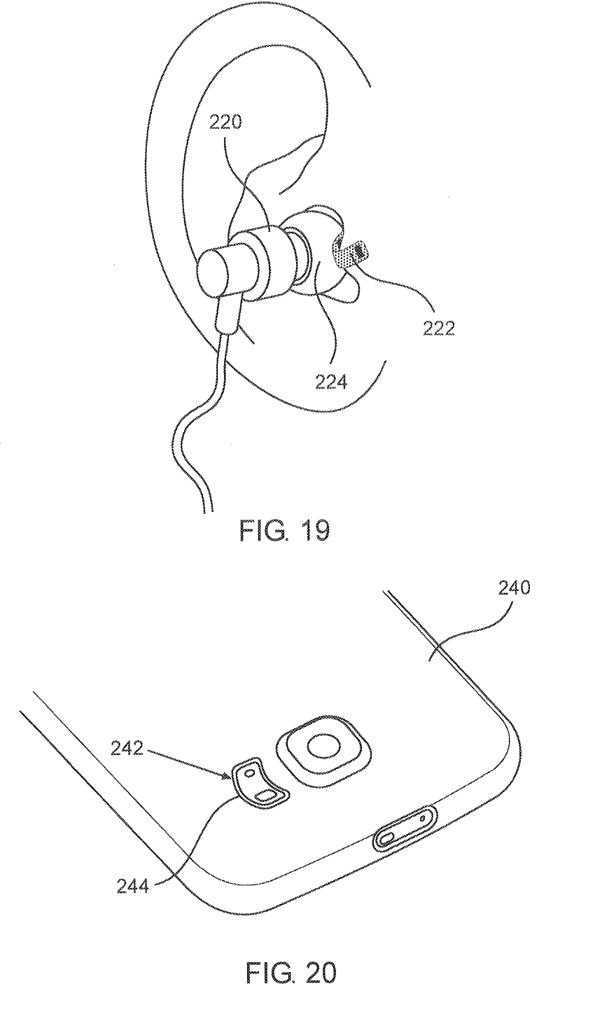

FIG. 19 schematically illustrates, according to an exemplary embodiment, a concave optical sensor 222 integrated with an earbud 220, further comprising an elastic member 224, for example a spring, positioned adjacent to the concave optical sensor 222 in a manner that presses the concave optical sensor 222 against the skin constantly and in a certain contact force.

FIG. 20 schematically illustrates, according to an exemplary embodiment, a concave optical sensor 242 integrated with a device 240 that is normally held by a user's palm, for example a portable cell phone, optionally comprising an elastic member 244, for example a spring, positioned adjacent to the concave optical sensor 242 in a manner that presses the concave optical sensor 242 against the skin constantly and in a certain contact force. The output data, e.g. oxygen saturation in blood may be displayed on a monitor of the computing device.

According to some embodiments, disclosed above, the elastic member, with or without the force limiter, is used with a concave optical sensor. However, the elastic member, with or without the force limiter, may be used also with an optical sensor in any shape and type known in the art, for example but not limited to, a flat optical sensor and a reflective optical sensor.

In the above description, an embodiment is an example or implementation of the subject matter. The various appearances of “one embodiment”, “an embodiment” or “some embodiments” do not necessarily all refer to the same embodiments.

Although various features of the subject matter may be described in the context of a single embodiment, the features may also be provided separately or in any suitable combination. Conversely, although the subject matter may be described herein in the context of separate embodiments for clarity, the subject matter may also be implemented in a single embodiment.

Embodiments may include features from different embodiments disclosed above, and embodiments may incorporate elements from other embodiments disclosed above. The disclosure of elements of the subject matter in the context of a specific embodiment is not to be taken as limiting their used in the specific embodiment alone.

Furthermore, it is to be understood that the subject matter can be carried out or practiced in various ways and can be implemented in embodiments other than the ones outlined in the description above. Meanings of technical and scientific terms used herein are to be commonly understood as by one of ordinary skill in the art to which the subject matter belongs, unless otherwise defined.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A concave optical sensor to be activated when worn on boney structure or a body part of a user, comprising:
at least one light source having at least one wavelength, wherein the light source is configured to emit light beams; and
at least one light receiver responsive to the light beams emitted from the light source,
wherein both the at least one light source and the at least one light receiver are positioned on a concave segment.

2. The sensor of claim 1, wherein the angle between the at least one light source and the at least one light receiver is different from 0 or 180 degrees.

3.-4. (canceled)

5. The sensor of claim 1, wherein the at least one light source comprises a single wavelength, and wherein a photoplethysmography (PPG) signal can be measured.

6. The sensor of claim 1, wherein the at least one light source includes at least two wavelengths, and wherein pulse oximetry can be measured.

7. The sensor of claim 6, wherein the sensor can be used to perform spectroscopic analysis of the blood constituents.

8. The sensor of claim 1, wherein the sensor is coupled to an external device, and wherein the external device is configured to allow calculations selected from the group consisting of pulse oximetry data and hemodynamic parameters.

9. (canceled)

10. The sensor of claim 1, wherein the sensor can be used to measure parameters selected from the group consisting of heart rate, pulse oximetry, and perfusion index.

11.-12. (canceled)

13. The sensor of claim 1, further comprising a display.

14. The sensor of claim 1, further comprising a communication module configured to allow wireless communication with external devices.

15. The sensor of claim 1, wherein parameters of the user can be measured or estimated wherein the parameters are selected from a group of parameters consisting of cardiovascular age, total health score, and life style wellness contribution.

16.-17. (canceled)

18. The sensor of claim 1, further comprising a biometric module, wherein physiological signals from the user are used to validate the user identity.

19. The sensor of claim 1, wherein the sensor is embedded within an object selected from the group consisting of a flat segment similar in shape to a credit card, a computerized device, a steering wheel, a piece of clothing, weighing scales, a sport training machine, a massaging device, an electrocardiogram (ECG) patch, and a patch that can temporarily be attached to the user's body.

20.-30. (canceled)

31. The sensor of claim 1, wherein the sensor is water proof.

32. A monitoring device comprising:
at least one concave optical sensor as claimed in claim 1; and
a microcontroller that is clipped over a device configured to at least partially wrap about a body part of the user.

33. The device of claim 32, wherein the device is a wearable device and the microcontroller is clipped on a strap over the body part.

34. The device of claim 32, wherein the microcontroller is strapped about a wrist or a finger of the user.

35. The device of claim 32, wherein the at least one concave sensor acts as a pulse oximeter.

36. A monitoring system comprising:
a concave optical sensor as claimed in claim 1;
a device coupled to the concave optical sensor;
a communication module configured to allow wireless transmission of data; and
a computing device configured to allow receiving signals from the communication module so as to perform a pulse wave analysis and derive hemodynamic parameters.

37. The monitoring system of claim 36, wherein said device is a wearable device that is selected from the group consisting of a ring, a watch, a wrist band, a combination thereof, and the like.

38.-44. (canceled)
